(19) United States
(12) Patent Application Publication
(10) Pub. No.: US 2016/0128590 A1
(43) Pub. Date: May 12, 2016

(54) ELECTRONIC APPARATUS HAVING MEASUREMENT ELECTRODES

(71) Applicant: KABUSHIKI KAISHA TOSHIBA, Tokyo (JP)

(72) Inventors: Minoru TAKIZAWA, Sagamihara Kanagawa (JP); Yasushi SASAKI, Mitaka Tokyo (JP); Meri UEDA, Yokohama Kanagawa (JP); Hiroshi OTA, Misato Saitama (JP)

(21) Appl. No.: 14/636,173

(22) Filed: Mar. 2, 2015

(30) Foreign Application Priority Data
Nov. 6, 2014 (JP) 2014-226430

(51) Int. Cl. A61B 5/0408; H01M 4/00

(52) U.S. Cl. CPC A61B 5/0408; H01M 4/00; A61B 2562/125; A61B 2562/0209

(57) ABSTRACT

A measurement device includes a housing, an electronic unit included in the housing and configured to measure a biological state of a subject, and an electrode to be placed on the subject for measuring the biological state. The electrode is embedded in the housing and has a surface that is exposed through an opening in the housing.

### CROSS-REFERENCE TO RELATED APPLICATION

This application is based upon and claims the benefit of priority from Japanese Patent Application No. 2014-226430, filed Nov. 6, 2014, the entire contents of which are incorporated herein by reference.

### FIELD

Embodiments described herein relate generally to an electronic apparatus having measurement electrodes.

### BACKGROUND

In the related art, a device detects a signal when an electrode exposed to a surface of a housing is attached to a subject.

### DESCRIPTION OF THE DRAWINGS

FIG. 1 is a perspective view of an electronic apparatus according to a first embodiment.

FIG. 2 is a plan view of a front surface of the electronic apparatus according to the first embodiment.

FIG. 3 is a plan view of aback surface of the electronic apparatus according to the first embodiment.

FIG. 4 is a cross-sectional view of the electronic apparatus taken along a line A-A in FIG. 2.

FIG. 5 is a cross-sectional view of the electronic apparatus taken along a line B-B in FIG. 2.

FIG. 6 is a cross-sectional view of an electronic apparatus according to the first embodiment in a manufacturing process using insert molding.

FIG. 7 is an explanatory diagram that shows flexibility of a housing of the electronic apparatus according to the first embodiment.

FIG. 8 is a perspective view of a substrate and a battery connected with each other in the electronic apparatus according to the first embodiment.

FIG. 9 is a plan view of a side (front surface side) of the substrate on which an electrode is disposed.

FIG. 10 is a plan view of a side (back surface side) of the substrate on which the battery is disposed.

FIG. 11 is a perspective view of an electrode of the electronic apparatus according to the first embodiment.

FIG. 12 is a perspective view of a modified example of an electrode of the electronic apparatus according to the first embodiment.

FIG. 13 is a perspective view of an substrate having a processed surface, on which the electrode is disposed.

FIG. 14 is a perspective view of a cradle that is used when the electronic apparatus according to the first embodiment is charged.

FIG. 15 is a front view of the cradle.

FIG. 16 is a rear view of the cradle.

FIG. 17 is a perspective view of a flexible substrate of an electronic apparatus according to a second embodiment.

FIG. 18 is a perspective view of a modified example of a flexible substrate of the electronic apparatus according to the second embodiment.

FIG. 19 is a perspective view of a flexible housing of the electronic apparatus according to the second embodiment.

FIG. 20 is a perspective view of an electronic apparatus according to a third embodiment, which includes an adhesive member and a holding tape.

FIG. 21 is a perspective view of an electronic apparatus according to a third embodiment, which includes an adhesive member and a holding tape.

FIG. 22 is a cross-sectional view of an electronic apparatus according to a fifth embodiment, which is chargeable through a non-touching charge.

FIG. 23 is a perspective view of an electronic apparatus according to a sixth embodiment, which has a charge terminal and a cover.

FIG. 24 is a perspective view of an electronic apparatus according to a seventh embodiment, which includes a first detector that measures pulse and a second detector that measures temperature of a body surface of a subject.

FIG. 25 is a perspective view of an electronic apparatus according to an eighth embodiment, in which an electrode and a housing are separated.

FIG. 26 illustrates a usage example of an electronic apparatus according to a ninth embodiment.

### DETAILED DESCRIPTION

An exemplary embodiment provides an electronic apparatus with fewer drawbacks.

In general, according to one embodiment, a measurement device includes a housing, an electronic unit included in the housing and configured to measure a biological state of a subject, and an electrode to be placed on the subject for measuring the biological state. The electrode is embedded in the housing and has a surface that is exposed through an opening in the housing.

A plurality of embodiments or modified examples that is exemplified in the following description includes the same configuration elements. Thus, in the following description, the same reference numerals and symbols are used for the same configuration elements, and repeated descriptions will be omitted.

### First Embodiment

An electronic apparatus 10 according to the present embodiment is, for example, a portable sensor unit that can detect an electrocardiographic potential. The electronic apparatus 10 includes a flat rectangular housing 12 having a surface 12a (sensor surface, top surface, first surface, first wall), a back surface 12b (charge surface, bottom surface, second surface, second wall), and side surfaces 12c, 12d, 12e, 12f (third surface, third wall). In addition, the housing 12 may have a shape, such as a polygonal shape, a circular shape, or an oval shape, when viewed from the surface 12a.

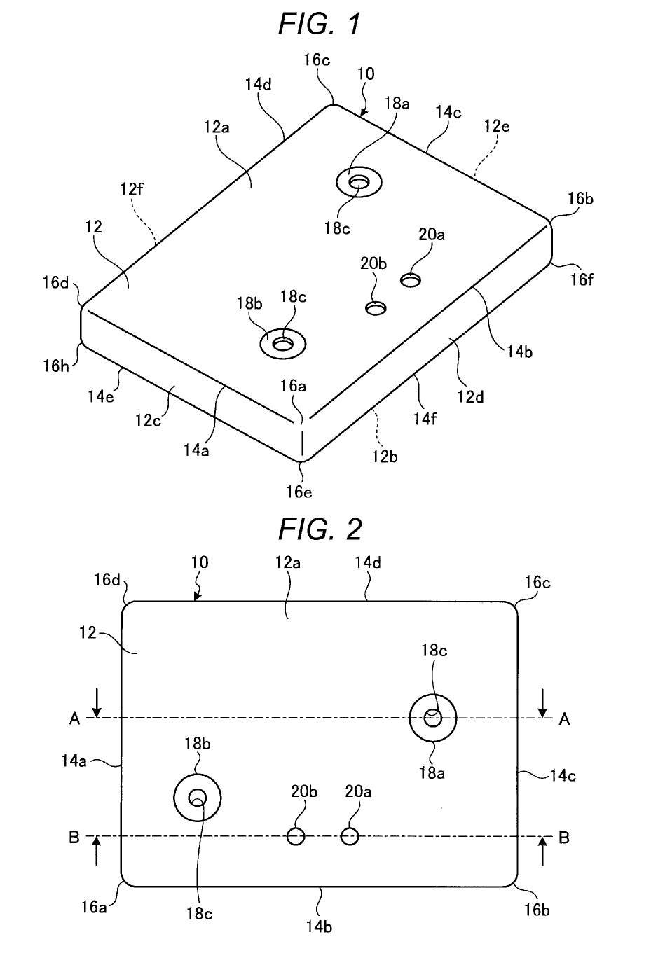

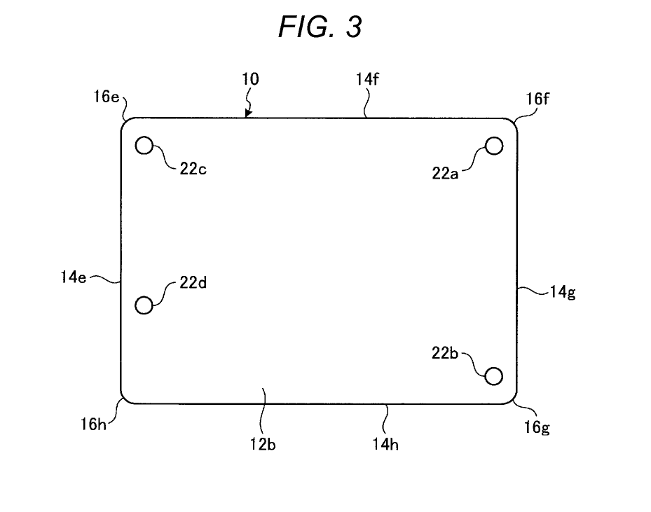

As illustrated in FIG. 1 to FIG. 3, sides 14a, 14b, 14c, and 14d (end portion, side portion, edge) of the surface 12a, and corners 16a, 16b, 16c, and 16d (apex, corner portion) at which the sides intersect respectively have a chamfered shape. In addition, sides 14e, 14f, 14g, and 14h (end portion, side portion, edge, refer to FIG. 3) of the back surface 12b, and corners 16e, 16f, 16g, and 16h at which the sides intersect respectively have a chamfered shape. In addition, a side in a thickness direction of the housing 12 between the corner 16a and the corner 16e, a side in a thickness direction of the housing 12 between the corner 16b and the corner 16f, a side in a thickness direction of the housing 12 between the corner 16c and the corner 16g, and a side in a thickness direction of the housing 12 between the corner 16d and the corner 16h, respectively have a chamfered shape. Due to this chamfered shape, hand touching feeling may be improved when a user holds the electronic apparatus 10. In addition, when the electronic apparatus 10 is used in a state of being mounted on a body surface, even if a hand is touched or an object is touched, the electronic apparatus 10 is hardly caught, and is suppressed from being dropped out due to the chamfered shape. In addition, the chamfered shape may be a curved chamfered shape or a flat chamfered shape.

As illustrated in FIG. 1 and FIG. 2 that is a flat view of the surface 12a, electrodes 18a and 18b (probes, terminals, metals, conductors) to be detected, which have detection surfaces (sensor surfaces, end portions, surfaces, one end surfaces) exposed from the surfaces 12a, are disposed on the surface 12a of the housing 12. The electrode 18a (first electrode) is, for example, a “positive electrode”, the electrode 18b (second electrode) is, for example, a “negative electrode,” and the electrodes 18a and 18b are disposed so as to be separated from each other. In addition, when the electronic apparatus 10 detects a biological signal (potential, electrocardiographic potential) for creating an electrocardiogram, in a case where a distance between the electrode 18a and the electrode 18b is equal to or greater than a predetermined distance, a stable detection result may be obtained. Meanwhile, as the electronic apparatus 10 is small, portability and ease of handling of the electronic apparatus 10 is improved. Therefore, in the present embodiment, the electrode 18a and the electrode 18b are disposed in a diagonal position to each other on the surface 12a, so that a predetermined distance between the electrode 18a and the electrode 18b is secured, and that a size of the electronic apparatus 10 is reduced.

As illustrated in FIG. 2, the electrode 18a is disposed in a position close to the corner 16c at which the side 14c and the side 14d intersect. Meanwhile, the electrode 18b is disposed in a position close to the corner 16a at which the side 14a and the side 14b intersect. In this way, by disposing the electrode 18a and the electrode 18b in a diagonal position to each other, it is possible to lengthen the distance between the electrode 18a and the electrode 18b, without increasing the size of the housing 12, compared to a case where the electrode 18a and the electrode 18b are disposed in a position in parallel with the side 14b or in a position in parallel with the side 14a, for example.

In addition, though it will be described below, the housing 12 has flexibility (softness) and may be bent. For example, the housing 12 may be bent such that a generatrix is generated in a direction in which the sides 14b and 14d in a longitudinal direction of the housing 12 intersect. Then, the electrode 18a is disposed in a position close to the corner 16c, that is, disposed in one end side in a longitudinal direction of the housing 12, and the electrode 18b is disposed in a position close to the corner 16a, that is, disposed in the other end side in a longitudinal direction of the housing 12. As a result, when the electronic apparatus 10 is touched to a body surface having a curved surface, adhesion of the electrode 18a and the electrode 18b, which exist in both end positions in the longitudinal direction, to the body surface may be improved by the curve of the housing 12.

In addition, though it will be described below, in order to further improve the adhesion of the electrode 18a and the electrode 18b to the body surface, a conductive adhesive member (gel member) may be disposed between the electrodes 18a and 18b and the body surface. In this way, it is considered that the adhesive member is relatively easily deformed, and thus by a disposed state, elapsed time, or the like, for example, an adhesive member of the electrode 18a (positive electrode) and an adhesive member of the electrode 18b (negative electrode) are electrically connected by being in contact with each other, or are electrically connected by sweat or the like which come out of the body surface, whereby a biological signal is not detected. In order to avoid such inconvenience, it is preferable that a distance between the electrode 18a and the electrode 18b be set to be long. In addition, for example, by disposing the electrode 18a in a position close to the corner 16b and disposing the electrode 18b in a position close to the corner 16d, diagonal position of the electrodes 18a and 18b may be disposed at diagonal positions.

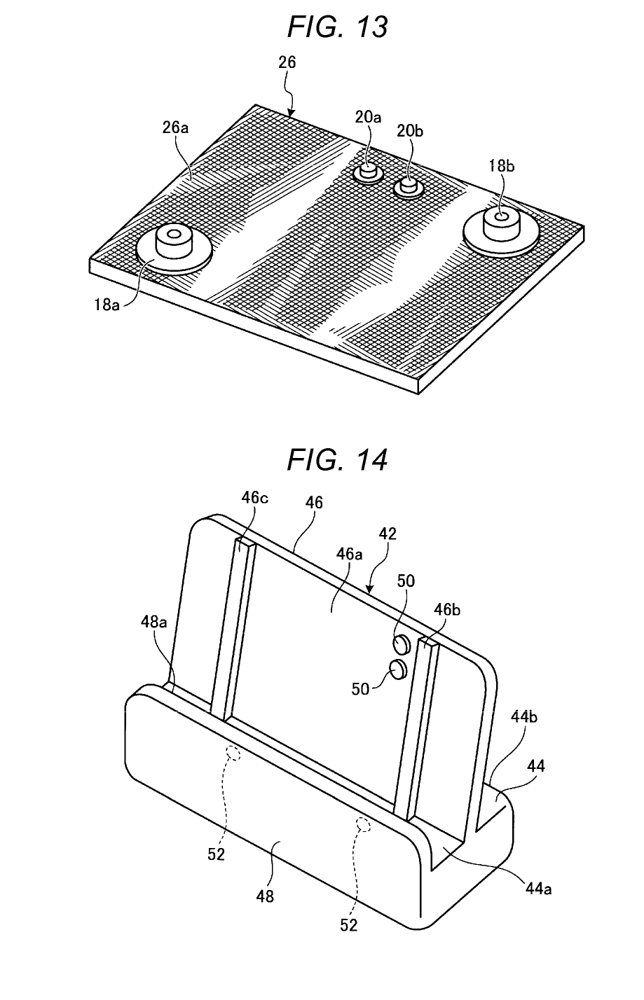

In addition, as illustrated in FIG. 1 and FIG. 2, on the surface 12a, data input and output terminals 20a and 20b (connectors, contact points, electrodes, metals, conductors) are disposed in a state of being exposed. For example, the input and output terminals 20a and 20b may be used for a case where a detected value that the electronic apparatus 10 acquires, data based on the detected value, information, or the like is transferred to an external apparatus using a wire method, a case where updating of software for controlling the electronic apparatus 10 is performed using a wire method, or the like, and may be electrically connected to a terminal of a dedicated adapter apparatus such as a cradle, which will be described below. As illustrated in FIG. 1 and FIG. 2, for example, the input and output terminals 20a and 20b are disposed substantially in parallel with the side 14b in a position adjacent to the side 14b. The input and output terminals 20a and 20b are not used in a state where the electronic apparatus 10 is in contact with a subject. In addition, a current does not flow between the input and output terminal 20a and the input and output terminal 20b. Thus, the input and output terminals 20a and 20b need not to be disposed so as to be separated, like as the electrodes 18a and 18b, and may be disposed in a state of being relatively approached. In addition, the input and output terminals 20a and 20b may also be disposed in any position of the electronic apparatus 10, but as will be described below, it is possible to increase an assembly efficiency by mounting the electrodes 18a and 18b and the input and output terminals 20a and 20b (refer to FIG. 13) that respectively have a similar shape on the same surface of a substrate.

As illustrated in FIG. 3, on the back surface 12b of the housing 12, a plurality of terminals 22a to 22d (connectors, contact points, terminals, metals, conductors) are disposed in a state of being exposed in the back surface 12b. In the present embodiment, a terminal 22a is exposed in a position adjacent to the corner 16f at which the side 14g and the side 14f intersect. In addition, a terminal 22b is exposed in a position adjacent to the corner 16g at which the side 14g and the side 14h intersect. In addition, a terminal 22c is exposed in a position adjacent to the corner 16e at which the side 14f and the side 14e intersect. Furthermore, a terminal 22d is exposed in a position apart in a direction of the corner 16e from the corner 16h at which the side 14e and the side 14h intersect. The terminals 22a to 22d may be used for various purposes. For example, the terminals 22a to 22d may be used as a terminal for charging a battery that is mounted in the electronic apparatus 10. In addition, the terminals 22a to 22d may also be used as terminals for data input and output in the same manner as the input and output terminals 20a and 20b. In addition, though it will be described below, when the housing 12 is formed by insert molding, the terminals 22a to 22d may also be used as a positioning member. In FIG. 3, as an example, the terminals 22a and 22b are configured so as to function as terminals for charging and as positioning members, and the terminals 22c and 22d are configured so as to function as positioning members.

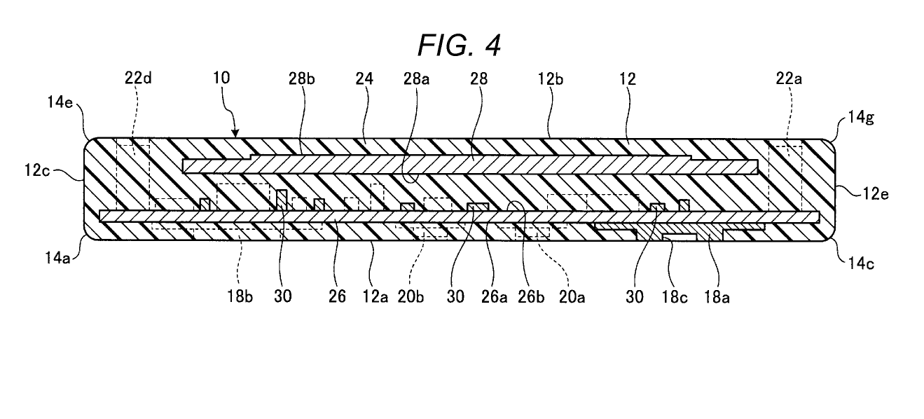

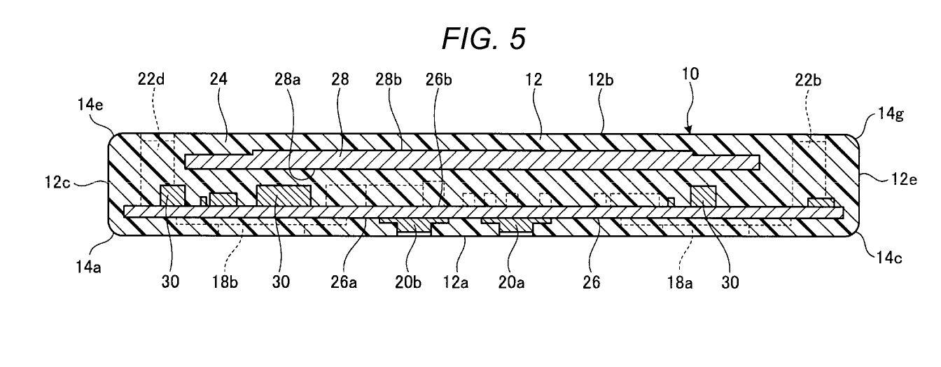

FIG. 4 is a cross-sectional view of the electronic apparatus 10 taken along a line A-A in FIG. 2, a cross section of which is viewed from the side 14d. FIG. 5 is a cross-sectional view of the electronic apparatus 10 taken along a line B-B in FIG. 2, a cross section of which is viewed from the side 14b. As illustrated in FIG. 4 and FIG. 5, in the electronic apparatus 10, the housing 12 is configured with a synthetic resin material 24 (silicone rubber, elastomer, flexibility resin) with flexibility (softness). For example, the housing 12 is molded in a state where a sub-assembly is buried within the housing 12, through insert molding that uses the sub-assembly of electronic components as a core. Then, inside the housing 12, a substrate 26 having a surface 26a (first substrate surface) on which the electrode 18a, the electrode 18b, the input and output terminals 20a, and the input and output terminal 20b are mounted so as to be exposed in the surface 12a of the housing 12, a battery 28 connected to the back surface 26b (second substrate surface) of the substrate 26, and a plurality of electronic components 30 (a reference numeral 30 is attached on behalf of a portion of electronic components in which a cross section appears) are housed. In addition, on the back surface 26b of the substrate 26, columnar terminals 22a to 22d are mounted so as to expose tips in the back surface 12b. In addition, the substrate 26 may be a printed board (rigid substrate) with high rigidity, and may be a flexible printed circuit (FPC) board with flexibility.

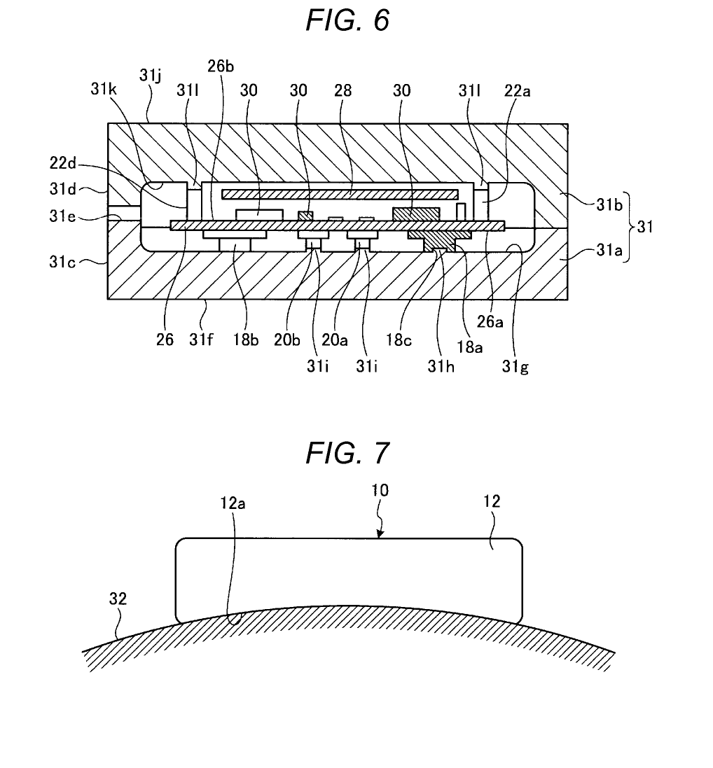

When the electronic apparatus 10 is manufactured through the above-described insert molding, the electrodes 18a and 18b, the input and output terminals 20a and 20b, the battery 28, and the sub-assembly of the plurality of electronic components 30 that are mounted on or supported by the surface 26a of the substrate 26, are housed inside an insert mold 31 that is formed of a first mold 31a (lower mold, fixed mold), and a second mold 31b (upper mold, lifting mold), as illustrated in FIG. 6. At an outer wall 31c (side surface wall) of the first mold 31a, a portion of an outer wall 31d of the second mold 31b, or a junction of the first mold 31a and the second mold 31b, an injection portion 31e is molded in order to fill the synthetic resin material 24 inside the insert mold 31.

In FIG. 6, as an example, an example in which the injection portion 31e is molded by the junction of the first mold 31a and the second mold 31b is illustrated. It is preferable that the injection portion 31e be set in a position in which the synthetic resin material 24 is easy to be filled according to mounting layout of the electronic components 30 on the substrate 26. As another example, in a substantially central portion in the longitudinal direction of the substrate 26, the injection portion 31e may be set in a position to which approximately ⅗ of the opening of the injection portion 31e is extended to the back surface 26b of the substrate 26 on which a filling amount of the synthetic resin material 24 is relatively large. In this way, by determining the position of the injection portion 31e, it is possible to improve a flow of the synthetic resin material 24 in the insert mold 31, and to decrease shrinkage of the synthetic resin material 24 or an occurrence rate of a void.

For example, an outer wall 31f (bottom surface wall) of the first mold 31a is fixed to a base portion of a molder (not illustrated), a heater, a cooling device, or the like is buried inside the first mold 31a, and temperature of the first mold 31a may be adjusted. In a predetermined position (position corresponding to a disposal relationship between the electrode 18a and the electrode 18b of the substrate 26) of an inner wall 31g (molded surface wall) of the first mold 31a, a convex portion 31h (pin, protrusion, positioning pin, difference in level, engaging portion) corresponding to a concave portion 18c (positioning portion, hole, recess, positioning hole, difference in level, engaging portion, refer to FIG. 1 and FIG. 2) formed on the exposed surfaces of the electrode 18a and the electrode 18b that are mounted on the substrate 26, is formed. That is, when the substrate 26 in which the electrode 18a and the electrode 18b are mounted is disposed in the inner wall 31g of the first mold 31a, the convex portion 31h and the concave portion 18c that are molded in the inner wall 31g are disposed so as to fit together. As a result, it is possible to perform smoothly and accurately positioning and supporting of the substrate 26 with respect to the inner wall 31g of the first mold 31a. In this way, in a state where the concave portions 18c of the electrode 18a and the electrode 18b are supported by the convex portion 31h, the synthetic resin material 24 is filled inside the insert molding mold 31. As a result, a difference in level between the tips of the electrodes 18a and the electrode 18b, and the surface 12a of the housing 12 formed of the synthetic resin material 24 is not generated. That is, it is possible to expose the electrode 18a and the electrode 18b from the surface 12a in a state where the biological signal (potential) can be reliably detected.

In addition, on the inner wall 31g of the first mold 31a, a convex portion 31i (protrusion, relief hole forming protrusion) is formed in a position corresponding to the input and output terminal 20a and the input and output terminal 20b that are mounted on the substrate 26 which is supported by the convex portion 31h through the electrode 18a and the electrode 18b. That is, when the housing 12 is completed, the tips of the input and output terminal 20a and the input and output terminal 20b may be exposed in a state of being recessed from the surface 12a of the housing 12. As a result, when the electronic apparatus 10 is mounted on the body surface of a subject or when the electronic apparatus 10 is handled, it is possible to suppress that something is in contact with the input and output terminal 20a and the input and output terminal 20b. In addition, when data is input and output, a terminal of an external apparatus may touch the input and output terminal 20a and the input and output terminal 20b by being inserted in the surface 12a of the housing 12. As a result, it is possible to form a structure in which an inflow or the like of noise is reliably suppressed during protection of a contact surface or data input and output.

An outer wall 31j (bottom surface wall) of the second mold 31b is fixed to a lifting portion of the molder (not illustrated). When the second mold 31b is apart from the first mold 31a, an insert component (substrate 26, battery 28, or the like) is mounted, or the molded electronic apparatus 10 is taken out. In addition, the second mold 31b approaches the first mold 31a and is combined together, and thereafter the housing 12 of the electronic apparatus 10 is molded by filling the synthetic resin material 24. In the same manner as the first mold 31a, a heater or a cooling device (not illustrated) is buried in the second mold 31b, and the second mold 31b is configured in such a manner that temperature thereof may be adjusted.

In addition, on an inner wall 31k of the second mold 31b, a convex portion 311 (protrusion, relief hole forming protrusion) is formed in a position corresponding to the terminals 22a to 22d (illustrated only as 22a and 22d in FIG. 6) that are mounted on the back surface 26b of the substrate 26, which is supported to the first mold 31a. That is, when manufacturing of the housing 12 is completed, the tips of the terminals 22a to 22d may be exposed in a state of being recessed from the back surface 12b of the housing 12. As a result, even when the electronic apparatus 10 is mounted on the body surface of a subject or when the electronic apparatus 10 is handled, an object may not be is in contact with the terminals 22a to 22d. In addition, in a state where the columnar terminals 22a to 22d are disposed on the back surface 12b of the substrate 26 and are in contact with the convex portion 311, the synthetic resin material 24 is filled. As a result, it is possible to accurately maintain a distance between the substrate 26 and the inner wall 31k of the second mold 31b. That is, it is possible to maintain more accurately a resin thickness of the synthetic resin material 24 from the back surface 26b of the substrate 26 to the back surface 12b of the housing 12, or a resin thickness of the synthetic resin material 24 from the battery 28 to the back surface 12b of the housing 12 to be a predetermined value.

In the present embodiment, as an example, four terminals 22a to 22d are mounted in the substrate 26, but the number of the terminals or an mounted position on the substrate 26 may be appropriately changed. In the present embodiment, as illustrated in FIG. 3, the terminals 22a to 22d are disposed so as to be positioned on approximately four corners of the back surface 12b of the housing 12. By performing such a disposal, it is possible to suppress variation of a resin thickness from the substrate 26 to the back surface 12b, or variation of a resin thickness from the battery 28 to the back surface 12b. In addition, as illustrated in FIG. 3, in the present embodiment, the terminal 22d is disposed in a deviated position so as to avoid other mounted components of the substrate 26. In addition, in another embodiment, for example, fifth terminal with the same shape as the terminal 22a may be disposed in a central portion of the substrate 26. In this case, the resin thickness may be further stabilized.

Returning to FIG. 4 and FIG. 5, an inner structure of the electronic apparatus 10 will be described. As illustrated in FIG. 4 and FIG. 5, the substrate 26 is disposed in a position closer to the surface 12a than the back surface 12b of the housing 12. By disposing the electrode 18a and the electrode 18b, and the input and output terminal 20a and the input and output terminal 20b having portions that has a relatively thin thickness are exposed on the housing 12, in the surface 26a of the substrate 26, it is possible to determine a distance between the surface 12a of the housing 12 and the surface 26a of the substrate 26, based on the thicknesses of such components. As a result, it is possible to decrease the entire thickness (distance between surface 12a and back surface 12b) of the electronic apparatus 10.

Meanwhile, on the back surface 26b of the substrate 26, a plurality of electronic components 30 are mounted, and the terminals 22a to 22d are mounted so as to surround electronic components 30. In addition, the battery 28 covers the mounting area of the plurality of electronic components 30. That is, the battery 28 is disposed in a position closer to the back surface 12b side than the surface 12a of the housing 12. In addition, the battery 28 is disposed in such a manner that a surface 28a is facing the back surface 26b (mounting surface of electronic components 30) of the substrate 26, and a back surface 28b is facing the back surface 12b of the housing 12.

Further, a distance between the back surface 28b of the battery 28 and the back surface 12b of the housing 12 is substantially equal to a distance between the surface 26a of the substrate 26 and the surface 12a of the housing 12. That is, since distances from the front and back of the housing 12 to the large components are substantially equal to each other, the flexibility (softness) of the housing 12 that has a thickness becomes the same as in the surface 12a side and the back surface 12b side, and when the electronic apparatus 10 is in hand, feeling becomes not extremely different in the front and the back.

In addition, in the surface 26a side (surface 12a side of the housing 12) of the substrate 26, not only the electrode 18a, the electrode 18b, the input and output terminal 20a, and the input and output terminal 20b are mounted, but also other electronic components 30 are not mounted. For this reason, for example, as illustrated in FIG. 7, when the electronic apparatus 10 is mounted on the body surface 32 of a subject, it is easy for the surface 12a of the housing 12 to be deformed, and to stick to the body surface 32. In addition, since it is easy for the housing 12 of the electronic apparatus 10 to be deformed, mounting feeling is improved.

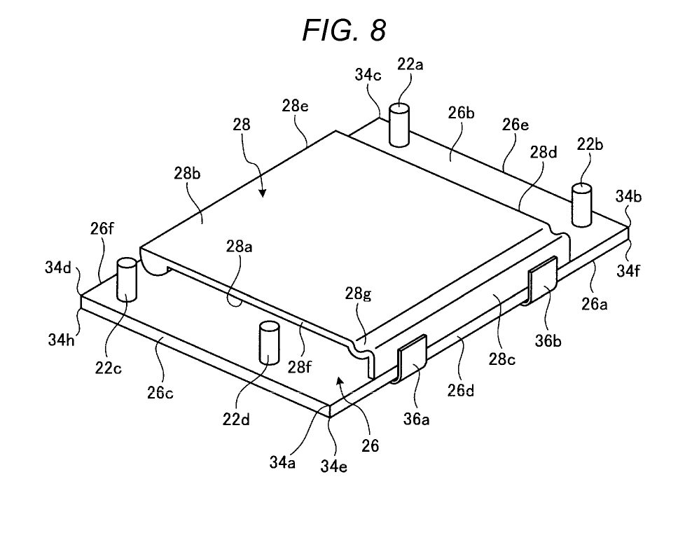

As illustrated in FIG. 4, FIG. 5, and FIG. 8 to FIG. 10, the terminal 22a is mounted in the vicinity of the corner 34c, at which the side surface 26e and the side surface 26f of the substrate 26 intersect, on the back surface 26b side. Similarly, the terminal 22b is mounted in the vicinity of the corner 34b, at which the side surface 26e and the side surface 26d of the substrate 26 intersect, on the back surface 26b side. The terminal 22c is mounted in the vicinity of the corner 34d, at which the side surface 26f and the side surface 26c of the substrate 26 intersect, on the back surface 26b side. In addition, the terminal 22d avoids the electronic component 38 that is mounted in the vicinity of corner 34a, at which the side surface 26c and the side surface 26d of the substrate 26 intersect, on the back surface 26b side, and is mounted in a position closer to the terminal 22c.

In addition, the electronic component 38 is a component that is preferable to be disposed on the corner of the substrate 26, and is, for example, an antenna component, specifically, a Bluetooth (registered trademark) chip including an antenna unit, or the like. In this way, the terminals 22a to 22d are disposed so as to enclose the electronic component 30 that is mounted on the substrate 26, or the battery 28.

In addition, as illustrated in FIG. 4 and FIG. 5, a height of the terminals 22a to 22d that are connected to the back surface 26b side of the substrate 26, in a direction (thickness direction of the substrate 26, thickness direction of the housing 12) perpendicular to the back surface 26b of the substrate 26 is set to be higher than a height of the plurality of electronic components 30 that are mounted on the substrate 26, or the height of the back surface 26b of the battery 28. According to this structure, the terminals 22a and 22d that are disposed on the four corners of the housing 12 function as a beam that maintains a shape of the housing 12, for example, even when the electronic apparatus 10 (housing 12) is dropped, or even when a large load is applied from the back surface 12b side of the housing 12. For example, when the electronic apparatus 10 is dropped, an impact load is applied to one of the terminals 22a to 22d, and it is possible to mitigate the impact of external force on the battery 28 or the electronic component 30. In addition, as illustrated in FIG. 4 and FIG. 5, the battery 28 is disposed so as to cover the plurality of electronic components 30. That is, the battery 28 is disposed so as to exert a function of suppressing a direct impact of the external force to the electronic components 30.

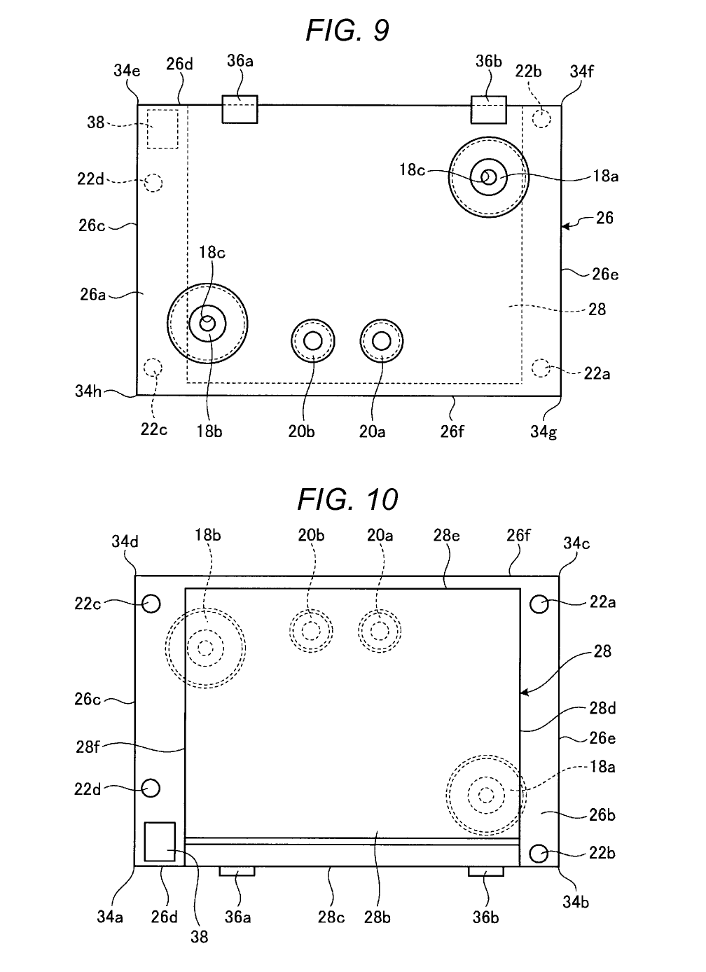

As illustrated in FIG. 8 to FIG. 10, in the plate-shaped battery 28, the surface 28a is facing the back surface 26b side of the substrate 26, and the back surface 28b is facing the back surface 12b of the housing 12. Then, the end portion 28c of a flat surface that is defined by the side 28d, the side 28e, and the side 28f is folded toward the substrate 26. In addition, a curvature portion 28g is formed in the folded portion, and if external force is applied from, for example, the back surface 12b side of the housing 12, the load of external force is mitigated by a spring effect of the curvature portion 28g. Connection plates 36a and 36b are provided in the end portion 28c, and are electrically connected to a power supply terminal (not illustrated), so as to go around the surface 26a of the substrate 26, as illustrated in FIG. 8 and FIG. 9. As the end portion 28c and the connection plates 36a and 36b support the battery 28 so as to be floated from the back surface 26b of the substrate 26, a mounting space of the electronic components 30 is formed between the substrate 26 and the battery 28, and a filling space of the synthetic resin material 24 may be formed. By filling the synthetic resin material 24 between the substrate 26 and the battery 28, an impact transferred to the electronic components 30 may be mitigated, when an impact applied to the electronic apparatus 10. Further, insulation of individual electronic component 30 and, a fixing of the electronic component 30 can be more reliably achieved.

In addition, as illustrated in FIG. 9, the electrode 18a is mounted in a position of the surface 26a side of the substrate 26 that is close to a corner 34f, at which the side surface 26d and the side surface 26e. Similarly, the electrode 18b is mounted in a position of the surface 26a side of the substrate 26 that is close to a corner 34h, at which the side surface 26c and the side surface 26d intersect and which is a diagonal position of the electrode 18a. Thus, even when an impact due to dropping or the like, or external force is applied from the surface 12a of the housing 12, it is possible to mitigate application of an impact to the substrate 26 to which the force is applied, as the electrode 18a and the electrode 18b are mounted in a diagonal position to each other.

In addition, also in the present embodiment, in a position of the surface 26a side of the substrate 26 that is close to the corner 34e, at which the side surface 26c and the side surface 26d of the substrate 26 intersect, and in a position of the surface 26a side of the substrate 26 that is close to the corner 34g, at which the side surface 26e and the side surface 26f of the substrate 26 intersect, a structure does not exist. Alternatively, by disposing a dummy support member such as a columnar member having the same height as, for example, the input and output terminals 20a and 20b, mitigation, absorption, or dispersion of the impact may be performed, when the external force is applied.

Here, biological signals that are detected by the electrodes 18a and 18b are retained in a storage unit that is mounted inside the electronic apparatus 10, and are transferred to an external apparatus such as an electrocardiogram output device (electrocardiograph, monitoring device, printing device) at a desired timing. In addition, the biological signals may also be transferred to the electrocardiogram output device, a portable terminal, or the like at a real time. As described above, the electronic apparatus 10 according to the present embodiment may transfer a biological signal or the like to an external apparatus using a wired method that uses the input and output terminals 20a and 20b. In addition, it is possible to transfer the biological signal to the external apparatus through the electronic component 38 (Bluetooth). In this case, the electrocardiogram may be monitored for 24 hours, for example. In addition, it is possible to perform a data transfer in a predetermined interval, a transfer in a desired timing, or an update of software in the electronic apparatus 10, through the Bluetooth (electronic component 38).

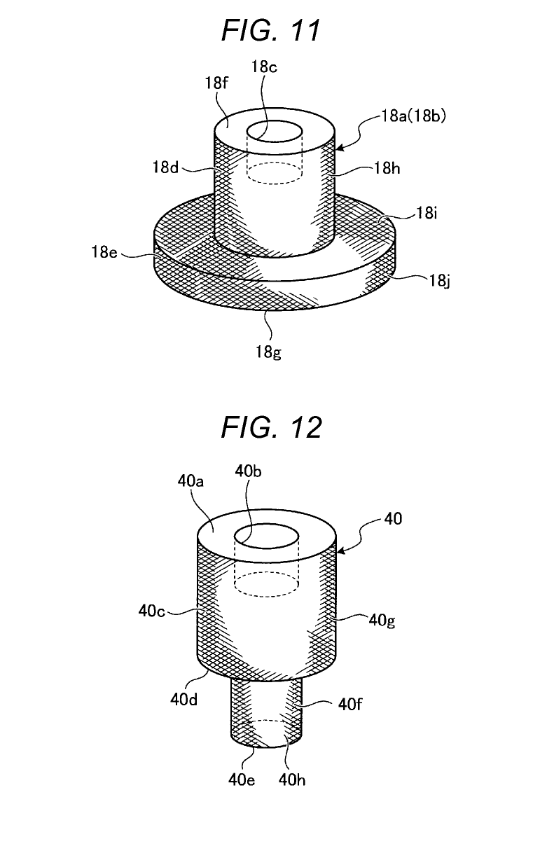

Next, a structure to improve adhesion of the synthetic resin material 24 that configure the housing 12 and each component will be described with reference to FIG. 11 to FIG. 13. FIG. 11 is a perspective view of the electrode 18a (18b). As illustrated in FIG. 11, the electrode 18a (18b) has a small diameter portion 18d in which a concave portion 18c that can be used for positioning is formed, and a large diameter portion 18e that is connected to a surface opposite to a surface on which the concave portion 18c is formed. That is, the small diameter portion 18d and the large diameter portion 18e forms a convex portion on a surface in contact with the housing 12. As a result, when the synthetic resin material 24 is filled in the vicinity of the electrode 18a (18b), the electrode 18a (18b) is pressed by the substrate 26 in which the electrode 18a (18b) is to be mounted, and it is possible to suppress that the electrode 18a (18b) from being detached from the substrate 26 and the housing 12.

In addition, as illustrated in FIG. 11, at least one of the concave portion and the convex portion is formed on the outer circumferential surface 18h of the small diameter portion 18d. For example, through surface-processing (for example, plasma processing, etching processing, mechanical processing) of the outer circumferential surface 18h, the concave portion or the convex portion is formed and a rough surface is formed. When the electrode 18a (18b) is in contact with the synthetic resin material 24, the contacted area is increased by forming the rough surface, and an anchor (hooking) effect is obtained.

In the same manner, the same surface processing is also performed on an upper surface 18i of the large diameter portion 18e or the outer circumferential surface 18j, and the anchor effect is obtained. That is, the surface processing is performed in the portions except the upper surface portion 18f, which is in contact with the body surface of a subject, and a lower surface portion 18g, which is electrically connected to the substrate 26. As a result, it is possible to increase adhesive strength of the electrode 18a (18b) and the synthetic resin material 24, and to reduce a gap or the like between the electrode 18a (18b) and the synthetic resin material 24. The reduction of the gap may contribute to improving a waterproof function or a dustproof function.

In addition, the surface processing may be performed in the entire portion of the outer circumferential surface 18h of the small diameter portion 18d, the outer circumferential surface 18j of the large diameter portion 18e, and an upper surface 18i, and may be selectively performed in a portion thereof. In addition, the rough surface (uneven shape) may have unevenness of, for example, a mesh shape, a lattice shape, or a dimple shape.

FIG. 12 is a modification example of the electrode 18a (18b) illustrated in FIG. 11, an electrode 40 has a large diameter portion 40c having a concave portion 40b which can be used for positioning of the above-described substrate 26 in an upper surface portion 40a, and a small diameter portion 40f, which is connected to a lower surface portion 40d of the large diameter portion 40c and in which a lower surface portion 40e is electrically connected to the substrate 26. That is, the small diameter portion 40f and the large diameter portion 40c forms a concave portion on a surface in contact with the housing 12. As a result, when the synthetic resin material 24 is filled in the vicinity of the electrode 40, the electrode 40 delivers an effect of pressing the synthetic resin material 24 between the electrode 40 and the substrate 26, and it is possible to prevent the synthetic resin material 24 from being peeled from the electrode 40.

In addition, as illustrated in FIG. 12, on the lower surface portion 40d or an outer circumferential surface 40g of the large diameter portion 40c, and on an outer circumferential surface 40h of the small diameter portion 40f, a rough surface of a concave portion or a convex portion is formed through surface processing in the same manner as in the electrode 18a (18b) illustrated in FIG. 11. Even in this case, when the electrode 40 is in contact with the synthetic resin material 24, a contact area with a surface of the electrode 40 is increased, whereby an anchor effect is obtained. That is, the surface processing is performed in the portions except an upper surface portion 40a that is in contact with the body surface of a subject, and a lower surface portion 40e that is electrically connected to the substrate 26. As a result, it is possible to increase adhesive strength of the electrode 40 and the synthetic resin material 24, and to reduce a gap or the like between the electrode 40 and the synthetic resin material 24. The reduction of the gap may contribute to improving a waterproof function or a dustproof function.

Here, the surface processing may be performed in the entire portion of the outer circumferential surface 40g and the lower surface portion 40d of the large diameter portion 40c, and the outer circumferential surface 40h of the small diameter portion 40f, and may be selectively performed in a portion thereof.

FIG. 13 shows an example in which at least one of the concave portion and the convex portion is provided on the surface 26a of the substrate 26, that is, a surface which is in contact with the housing 12 (synthetic resin material 24) and on which the electrodes 18a and 18b and the input and output terminals 20a and 20b are disposed. Even in this case, in the same manner as in the electrode 18a (18b) or the electrode 40, a concave portion or a convex portion is formed through surface-processing (for example, plasma processing, etching processing, mechanical processing) of the surface 26a, wand as a result a rough surface is formed. When the surface 26a of the substrate 26 is in contact with the synthetic resin material 24, the contacted area is increased by forming the rough surface, and an anchor effect is obtained. As a result, it is possible to suppress the synthetic resin material 24 (housing 12) from being floated (peeled) from the substrate 26.

By increasing adhesion of the synthetic resin material 24 of the housing 12 and each component, it is possible to suppress that moisture, dust, or the like from entering the electronic apparatus 10. Also, fixing of each component (substrate 26, battery 28, electronic component 30) may be performed using the synthetic resin material 24 having flexibility after being cured. As a result, even when an impact from outside is applied to the electronic apparatus 10, the impact is mitigated by the synthetic resin material 24, and it is possible to suppress each component from being damaged.

With reference to FIG. 14 to FIG. 16, an example of a cradle (expansion apparatus, external apparatus, adapter) 42 which may charge the electronic apparatus 10, and input and outputs data through the input and output terminals 20a and 20b is described. The cradle 42 has a base portion 44, a rear surface support portion 46, and a front surface support portion 48. On the base portion 44, the rear surface support portion 46 having a rectangular box shape and a plate shape extending along a side portion 44b in a longitudinal direction is provided in a form that is slightly inclined backward from the vertical, at an approximately center portion of the side portion 44a in a lateral direction. In addition, the front surface support portion 48 is provided on a front surface side of the base portion 44 in parallel with the rear surface support portion 46. An interval in a direction of the side portion 44a between the rear surface support portion 46 and the front surface support portion 48 corresponds to a thickness of the electronic apparatus 10 that is mounted on the cradle. The electronic apparatus 10 can be set between the rear surface support portion 46 and the front surface support portion 48, and supported thereby.

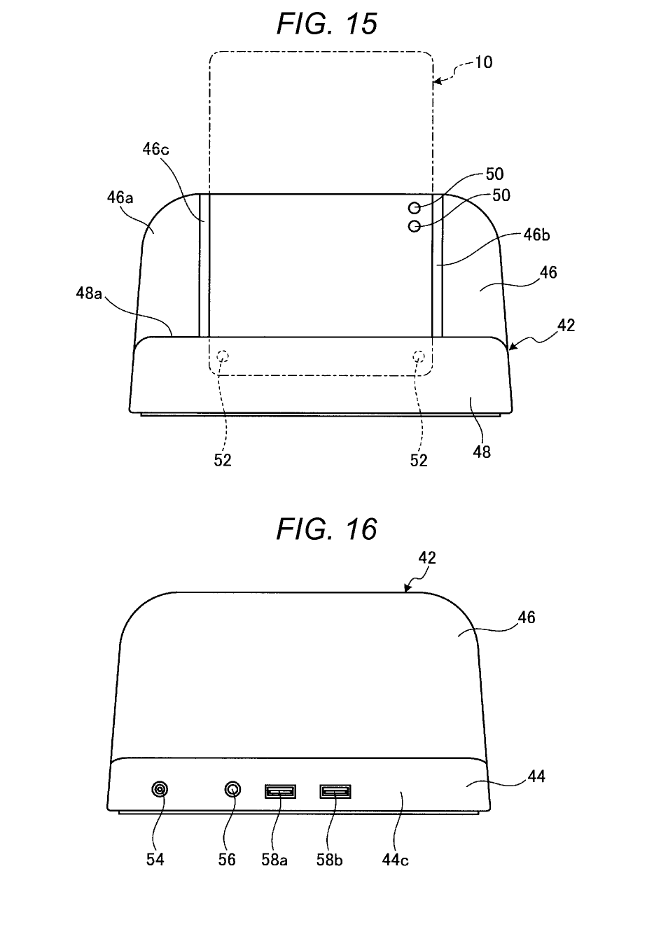

As illustrated in FIG. 15, on the surface 46a of the rear surface support portion 46, two guide members 46b and 46c are provided at an interval corresponding to a width (length of side portion 14e) in a lateral direction of the electronic apparatus 10. The electronic apparatus 10 is guided thereby when the apparatus is set in a predetermined position of the cradle 42. The electronic apparatus 10 is set such that the input and output terminals 20a and 20b (refer to FIG. 2) are in contact with connection terminals 50 n the surface 46a, so that the surface 12a of the housing 12 is facing the surface 46a.

The two connection terminals 50 are disposed so as to correspond to a mounting interval of the input and output terminals 20a and 20b. The connection terminals 50 are freely movable back and forth using, for example, a spring structure or the like. The connection terminals 50 are contactable with the input and output terminals 20a and 20b that are exposed in a concave manner from the surface 12a of the housing 12. A biological signal detected by the electronic apparatus 10 is transferred to an external apparatus by contact of the two, and it is possible to transfer update data of software towards the electronic apparatus 10.

In addition, if the electronic apparatus is inserted between the rear surface support portion 46 and the front surface support portion 48 of the cradle 42, such that the input and output terminals 20a and 20b of the electronic apparatus 10 are in contact with the connection terminals 50, the terminals 22a and 22b (refer to FIG. 3) of the electronic apparatus 10 are in contact with the power supply terminals 52 that is formed on an inner side surface 48a of the front surface support portion 48 facing the rear surface support portion 46. The power supply terminals 52 are disposed in an interval corresponding to the mounting interval of the terminals 22a and 22b. The power supply terminals 52 are freely movable back and forth by, for example, a spring structure or the like, and contactable with the terminals 22a and 22b that are exposed in a concave manner from the back surface 12b of the housing 12. As a result, when the power supply terminals 52 are in contact with the battery, the battery 28 can be charged.

As illustrated in FIG. 16, on the back surface of the rear surface support portion 46, that is, a back surface 44c of the base portion 44, jack 54 for AC adapter that inputs an external power to the power supply terminal 52, a connection jack 56 to which an external apparatus, such as an electrocardiograph or a monitor is connected, USB terminals 58a and 58b to which USB cables are connected, or the like are disposed. In addition, when the electronic apparatus 10 is mounted on the cradle 42, data transmission and reception through the connection terminals 50, or power supply through the power supply terminals 52 may be automatically performed, and a desired operation may be performed by a switch or the like.

In this way, in the electronic apparatus 10 according to the present embodiment, the electrodes 18a and 18b, the input and output terminals 20a and 20b, the terminals 22a to 22d, the substrate 26, the battery 28, and each of the plurality of electronic components 30 are covered with the synthetic resin material 24 formed through an insert molding and in contact with the synthetic resin material 24 (buried in synthetic resin material 24). As a result, waterproof property and dustproof property of the electrodes 18a and 18b, the input and output terminals 20a and 20b, the terminals 22a to 22d, the substrate 26, the battery 28, the plurality of electronic components 30, and the like are satisfactorily obtained.

In addition, since the housing 12 is formed by molding of the synthetic resin material 24 through the insert molding, deformation or breakage of the housing 12 hardly occurs. In this point as well, waterproof property and dustproof property can be improved.

In addition, since the electrodes 18a and 18b, the input and output terminals 20a and 20b, the terminals 22a to 22d, the substrate 26, the battery 28, and the plurality of electronic components 30 are respectively covered with the synthetic resin material 24, even when an impact is applied from outside, the impact is mitigated and damage is decreased.

In addition, since the housing 12, which defines an outer shell of the electronic apparatus 10, is molded with the synthetic resin material 24 having flexibility, texture during being in contact with the body surface of a subject, or hand feeling during being handled is improved, and usability of the electronic apparatus 10 is improved.

### Second Embodiment

As illustrated in the first embodiment described above, as the electronic apparatus 10 is mounted on the body surface of a subject, it is desirable that the housing 12 have a certain flexibility (softness). Meanwhile, when the housing 12 is too soft, the electronic apparatus 10 (housing 12) may be unnecessarily bent and external force stronger than necessary may be applied. For example, the electronic component 30 or the like, which is mounted on and electrically connected to the substrate 26, may cause a contact failure. Therefore, the electronic apparatus 10 according to the present embodiment has some portion with flexibility and the other portion that has rigidity higher than the portion, in order to improve mounting feeling and handling property.

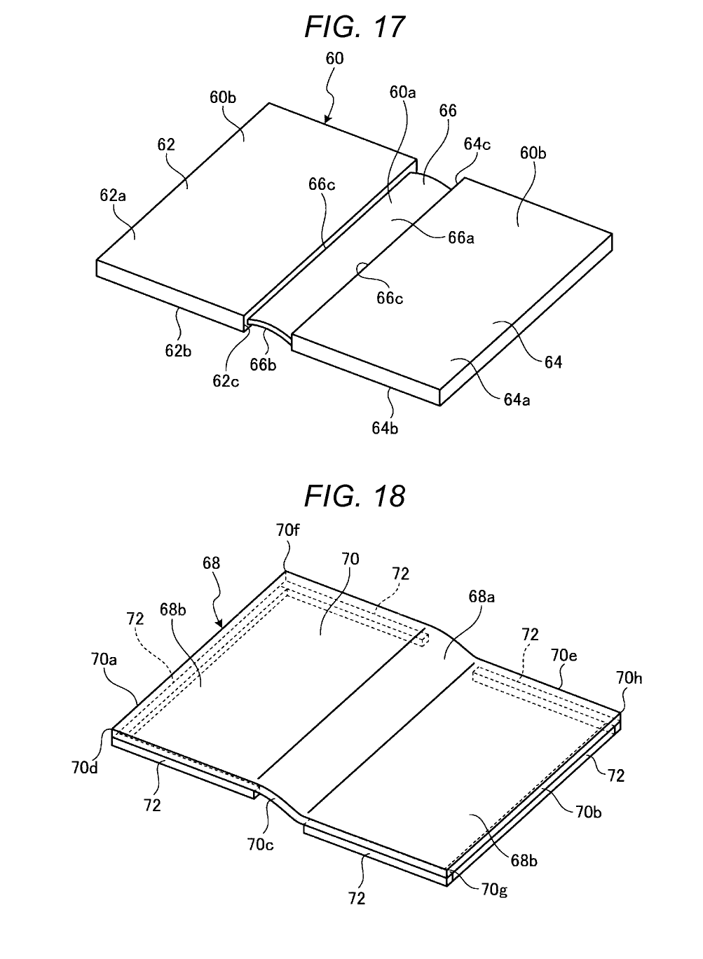

FIG. 17 is a perspective view of a substrate 60 in which flexibility of the electronic apparatus 10 may be adjusted. In the substrate 60, so that at least a portion is bendable, rigid substrates 62 and 64 using an insulating substrate without flexibility, and a flexible printing substrate 66 are combined. The rigid substrate 62 includes a surface 62a on which, for example, the electrode 18a and the input and output terminal 20a (refer to FIG. 9) are mounted, and a rear surface 62b on which a portion of the electronic components 30 is mounted.

In addition, the rigid substrate 64 includes a surface 64a on which the electrode 18b and the input and output terminal 20b (refer to FIG. 9) are mounted, and a rear surface 64b on which the rest of the electronic components 30 is mounted. In addition, in FIG. 17, since the surface 66a and the rear surface 66b of the flexible printing substrate 66 are a first area portion 60a which may be bent, mounted components are not mounted by only wiring pattern (not illustrated).

In the rigid substrate 62 and the rigid substrate 64, the surfaces 62a and 64a, and the rear surfaces 62b and 64b are rectangular shapes, respectively, and the flexible printing substrate 66 having a side surface 66c with the same length as that of a side surface 62c is mechanically connected to the side surface 62c in a longitudinal direction. A wiring pattern formed on the surface 66a of the flexible printing substrate 66 is electrically connected to a wiring pattern (not illustrated) of the surface 62a of the rigid substrate 62. In the same manner, a wiring pattern formed on the rear surface 66b of the flexible printing substrate 66 is electrically connected to a wiring pattern (not illustrated) of the rear surface 62b of the rigid substrate 62.

In addition, in the flexible printing substrate 66, the side surface 66c connected to the rigid substrate 62 and the side surface 66c opposite to that are mechanically connected to the side surface 64c in a longitudinal direction of the rigid substrate 64, and the flexible printing substrate 66 is electrically connected to the wiring pattern of the rigid substrate 64. That is, the rigid substrate 62, the flexible printing substrate 66, and the rigid substrate 64 function as a series of substrate, and have the first area portion 60a that may be bent, and a second area portion 60b having rigidity higher than the first area portion 60a.

In addition, when the substrate 60 is used, a battery that is mounted on the substrate 60 preferably has a structure in which a portion corresponding to the flexible printing substrate 66 may be bent, or is divided into one battery for the rigid substrate 62 and the other battery for the rigid substrate 64. In addition, the battery may be mounted on one of the rigid substrate 62 and the rigid substrate 64.

Here, the substrate 60 is used instead of the substrate 26 illustrated in FIG. 4, FIG. 5, and the like, and the electrodes 18a and 18b, the input and output terminals 20a and 20b, the electronic components 30, and the like are disposed thereon. According to such a structure, it is possible to obtain the electronic apparatus 10 having an area which may be bent and pressing-deformed and an area which may be pressing-deformed. That is, in the first area portion 60a which is configured in the flexible printing substrate 66, the electronic apparatus 10 is bent or the electronic apparatus 10 is deformed according to a body surface, and the flexibility of the synthetic resin material 24 may be used for improving adhesion. In addition, in the second area portion 60b which is configured in the rigid substrate 62 and 64, the electronic apparatus 10 is deformed according to a body surface, and the flexibility of the synthetic resin material 24 may be used for improving adhesion.

In addition, an example in which the side surface 66c of the flexible printing substrate 66 is connected to the side surface 62c (64c) of the rigid substrate 62 (rigid substrate 64), in the substrate 60 illustrated in FIG. 17 is illustrated, but it is possible to obtain the same effects also with a structure in which a portion of the rear surface 66b of the flexible printing substrate 66 is fixed to a portion of the surface 62a (64a) of the rigid substrate 62 (64).

In the example of FIG. 17, the flexible printing substrate 66 is used in one place, but by using a plurality of flexible printing substrates 66, it is possible to configure the electronic apparatus 10 with a higher flexibility. In addition, by changing a disposal direction of the flexible printing substrate 66 to 90 degrees, it is possible to manufacture the electronic apparatus 10 with a different bending direction.

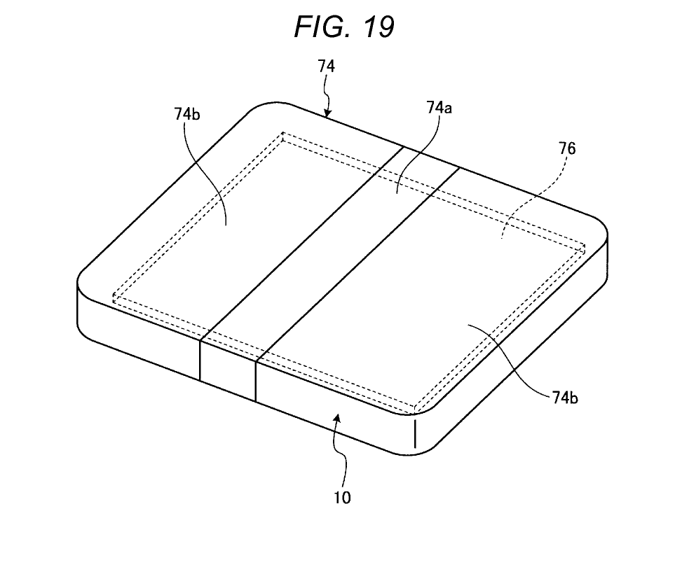

FIG. 18 is a perspective view of a substrate 68 of another structure example in which flexibility of the electronic apparatus 10 may be adjusted. The substrate 68 is configured with a flexible printing substrate 70. Further, the substrate 68 includes a first area portion 68a that enables the flexible printing substrate 70 to be bent and a second area portion 68b including a reinforcing member 72 which prevents the bending of the flexible printing substrate 70. The reinforcing member 72 may be formed of, for example, a metal or a resin, of which rigidity is adjusted. For example, the reinforcing member 72 is fixed along sides 70a and 70b in a lateral direction of the flexible printing substrate 70.

In addition, the reinforcing member 72 is fixed in a portion of the flexible printing substrate 70 corresponding to the second area portion 68b, which includes a corner 70d in a side 70c in a longitudinal direction of the flexible printing substrate 70. In the same manner, the reinforcing member 72 is fixed in a portion of the flexible printing substrate 70 corresponding to the second area portion 68b, which includes a corner 70f in a side 70e in a longitudinal direction of the flexible printing substrate 70. In addition, the reinforcing member 72 is fixed in a portion of the flexible printing substrate 70 corresponding to the second area portion 68b including a corner 70g in the side 70c in a longitudinal direction of the flexible printing substrate 70. In the same manner, the reinforcing member 72 is fixed in a portion of the flexible printing substrate 70 corresponding to the second area portion 68b including a corner 70h in a side 70e in a longitudinal direction of the flexible printing substrate 70.

In this way, by fixing the reinforcing member 72 in a portion of the flexible printing substrate 70, the second area portion 68b that prevents the bending of the flexible printing substrate 70 may be formed. In addition, a portion that does not fix the flexible printing substrate 70 becomes the first area portion 68a that enables the flexible printing substrate 70 to be bent. In addition, the disposal and handling of the electrodes 18a and 18b, the input and output terminals 20a and 20b, the electronic components 30, and the battery in the substrate 68 are the same as those of the substrate 60 illustrated in FIG. 17.

In the substrate 68 configured in this way, in the same manner as the substrate 60 illustrated in FIG. 17, the electronic apparatus 10 is bent in the first area portion 68a, or the electronic apparatus 10 is deformed according to a body surface, and the flexibility of the synthetic resin material 24 may be used for improving adhesion. In addition, the electronic apparatus 10 is deformed according to a body surface in the second area portion 68b, and the flexibility of the synthetic resin material 24 may be used for improving adhesion.

In the example of FIG. 18, the first area portion 68a in which the reinforcing member 72 is not disposed is used as one place. Alternatively, by using a plurality of portions in which the first area portions 68a are formed, it is possible to configure the electronic apparatus 10 with a higher flexibility. In addition, by appropriately selecting the position of the first area portion 68a, it is possible to manufacture the electronic apparatus 10 which may be bent in a desired position.

FIG. 19 is a perspective view illustrating an example of an electronic apparatus 10 in which adjustment of the flexibility of the electronic apparatus 10 is obtained through the structure of a housing 74. The housing 74 includes a flexible printing substrate 76 therein. The housing 74 has a first housing area portion 74a that enables the flexible printing substrate 76 to be bent and second housing area portions 74b that has rigidity higher than that of the first housing area portion 74a and prevents the bending of the flexible printing substrate 76. That is, the first housing area portion 74a is molded by a synthetic resin material with high flexibility, and the second housing area portion 74b is molded by a synthetic resin material with lower flexibility (stronger rigidity) than that of the first housing area portion 74a.

According to this configuration, when the electronic apparatus 10 is manufactured through insert molding, the first housing area portion 74a and the second housing area portion 74b may be easily molded through a so-called two color molding. Through the two color molding, an area in which the flexible printing substrate 70 may be bent and another area in which the flexible printing substrate 70 is hardly bent are molded. In the electronic apparatus 10 having the housing 74 configured in this way, the first housing area portion 74a functions so as to bend the electronic apparatus 10.

In the example of FIG. 19 the first housing area portion 74a is used as one place. By using a plurality of portions in which the first housing area portion 74a is formed, it is possible to configure the electronic apparatus 10 with a higher flexibility. In addition, by appropriately selecting the position of the first housing area portion 74a, it is possible to manufacture the electronic apparatus 10 which may be bent in a desired position.

### Third Embodiment

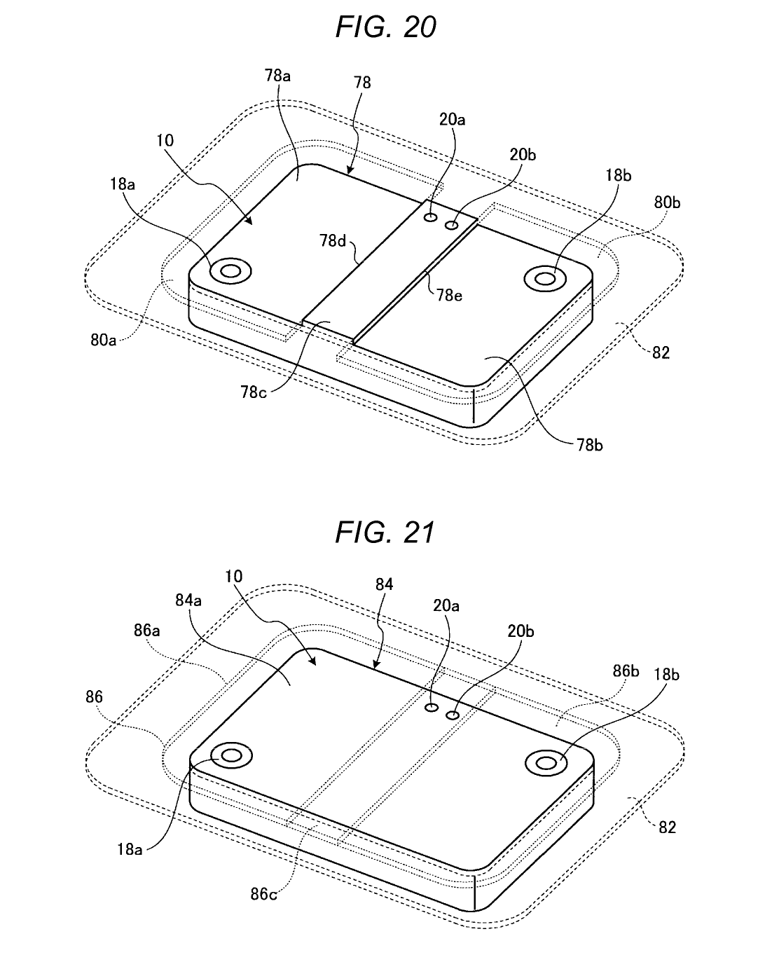

With reference to FIG. 20, a housing 78 with a structure that is suitable to stick to the body surface of a subject for detecting a biological signal will be described. A basic structure or function of the electronic apparatus 10 including the housing 78 is the same as those of the electronic apparatus 10 illustrated in FIG. 1 or the like described above. In addition, the housing 78 has a structure so that a first adhesion member 80a with conductivity and a second adhesion member 80b with conductivity are easily mounted, in order to stabilize sticking and fixing to the body surface of a subject. The first adhesion member 80a and the second adhesion member 80b have adhesion in a state of gel, and contain electrolyte in gel, have ion conductivity, and achieve a conductive function. The first adhesion member 80a and the second adhesion member 80b have adhesion in both sides, and one side thereof is adhered to a first support area portion 78a (second support area portion 78b) and fixed. Here, the support area portion is referred to as, for example, a support portion, a support area, or the like. In addition, the other sides of the first adhesion member 80a and the second adhesion member 80b are stuck to a body surface to fix the housing 78 to the body surface. In addition, areas of the first adhesion member 80a and the second adhesion member 80b may be formed widely more than areas of the first support area portion 78a and the second support area portion 78b, and a fixed strength of the housing 78 to the body surface may be increased.

In the same manner as the electronic apparatus 10 described above, even when the housing 78 is used, the electrode 18a and the electrode 18b are required to be insulated, in order to detect a biological signal. That is, the first adhesion member 80a and the second adhesion member 80b are required to be insulated. For example, when the first adhesion member 80a and the second adhesion member 80b are adhered to the electronic apparatus 10 including the housing 12 with a flat surface 12a illustrated in FIG. 1, the first adhesion member 80a and the second adhesion member 80b are required to be mounted in a state of being separated. However, when the first adhesion member 80a and the second adhesion member 80b are mounted with an interval on the flat surface 12a, the first adhesion member 80a and the second adhesion member 80b may be deformed and be in contact with each other when they are pressed to the body surface to attach the electronic apparatus 10 to the subject. In addition, the first adhesion member 80a may be electrically connected to the second adhesion member 80b through the sweat that comes out of the body surface. In view of this possibility, the first adhesion member 80a and the second adhesion member 80b are required to be disposed with a sufficient interval. In addition, when the first adhesion member 80a and the second adhesion member 80b are disposed in this way, the interval portion becomes concave, and, thus, when the electronic apparatus 10 is mounted on a body surface, the interval may cause uncomfortable feeling.

The housing 78 has a structure for decreasing conductivity caused by the deformation or the sweat, and uncomfortable feeling, described above. As illustrated in FIG. 20, the housing 78 includes the first support area portion 78a in which the electrode 18a is exposed and the second support area portion 78b in which the electrode 18b is exposed. In addition, the housing includes a protrusion portion 78c that is disposed between the first support area portion 78a and the second support area portion 78b, and protrudes more than the first support area portion 78a and the second support area portion 78b. The input and output terminals 20a and 20b are exposed in the protrusion portion 78c. Here, it is desirable that an amount of protrusion of the protrusion portion 78c from the first support area portion 78a and the second support area portion 78b be set as a thickness corresponding to the thicknesses of the first adhesion member 80a and the second adhesion member 80b that are mounted, for example.

In this way, by forming the protrusion portion 78c that protrudes more than the first support area portion 78a and the second support area portion 78b on the surface of the housing 78, the protrusion portion 78c becomes a guide for positioning when the first adhesion member 80a and the second adhesion member 80b are mounted on the housing 78. That is, by using a side surface 78d of the protrusion portion 78c in a longitudinal direction as a guide when the first adhesion member 80a is mounted, the first adhesion member 80a may be mounted in a predetermined position of the housing 78. In the same manner, by using a side surface 78e of the protrusion portion 78c in a longitudinal direction as a guide when the second adhesion member 80b is mounted, the second adhesion member 80b may be mounted in a predetermined position of the housing 78. Furthermore, by the protrusion portion 78c, the first adhesion member 80a and the second adhesion member 80b may be separated substantially, and thus it is possible to suppress an electric conduct caused by deformation of the first adhesion member 80a and the second adhesion member 80b. In addition, it is possible to decrease the conductivity of the first adhesion member 80a and the second adhesion member 80b due to sweat. In addition, by pasting a sheet having water absorbing property, such as non-woven fabric on the protrusion portion 78c, it is possible to further decrease generation of conductivity caused by sweat.

In addition, in the example of FIG. 20, fixing of the electronic apparatus 10 including the housing 78 to a body surface may be performed using the first adhesion member 80a and the second adhesion member 80b. In order to secure more reliable adhesion, in the example of FIG. 20, a retention tape (cover member) 82 with adhesion is further used, which is obtained by cutting out an external portion of the housing 78, that is, a portion on a rear surface that is opposite to a surface facing the electrode 18a and 18b. By using the retention tape 82, the first adhesion member 80a that protrudes long in an outer circumferential direction from the first support area portion 78a, or the second adhesion member 80b that protrudes long in an outer circumferential direction from the second support area portion 78b are slightly adhered. As a result, the retention tape 82 may further increase the fixed strength of the housing 78 to a body surface.

In addition, the retention tape 82 may be configured with non-woven fabric or the like, and may be a tape with large size that covers all of the housing 78, the first adhesion member 80a, which is protrudes long in an outer circumference of the housing 78, and the second adhesion member 80b. In this case, by attaching the retention tape 82 to a body surface so as to cover the housing 78, it is possible to more reliably perform fixing of the electronic apparatus 10.

### Fourth Embodiment

The electronic apparatus 10 illustrated in FIG. 21 includes a housing 84 having a flat surface 84a (sensor surface, top surface, first surface, first wall) similarly to that of the electronic apparatus 10 illustrated in FIG. 1. Then, a conductive adhesion member 86 with adhesion on both sides that are adhered to the surface 84a in which the electrodes 18a and 18b and the input and output terminals 20a and 20b of the housing 84 are exposed, has a first adhesion member 86a, a second adhesion member 86b, and a third adhesion member 86c. The first adhesion member 86a is an adhesion member with conductivity and covers the electrode 18a. The second adhesion member 86b is an adhesion member with conductivity and covers the electrode 18b. Meanwhile, the third adhesion member 86c is an adhesion member with non-conductivity and covers the input and output terminals 20a and 20b.

The adhesion member 86 is configured with one sheet in which the adhesion members are disposed in the sequence of the first adhesion member 86a, the third adhesion member 86c, and the second adhesion member 86b, so that the adhesion member 86 may be adhered to the surface 84a of the housing 84 and be mounted. In addition, the third adhesion member 86c with non-conductivity is disposed between the first adhesion member 86a and the second adhesion member 86b, and thus it is possible to reliably perform insulation of the electrode 18a and the electrode 18b. Furthermore, even when sweat comes out of a body surface, since the third adhesion member 86c with non-conductivity is disposed, it is possible to decrease a possibility that the first adhesion member 86a and the second adhesion member 86b (electrode 18a and electrode 18b) is electrically conducted. In addition, the first adhesion member 86a, the second adhesion member 86b, and the third adhesion member 86c may be configured as one sheet as described above, and each may be separated. In addition, the third adhesion member 86c may be integrated with either of the first adhesion member 86a and the second adhesion member 86b.

In addition, as illustrated in FIG. 21, an area of the adhesion member 86 may be formed largely more than an area of the surface 84a of the housing 84, and an adhesive strength of the housing 84 (electronic apparatus 10) with respect to a body surface may be increased. In addition, the retention tape 82 may also be mounted on the housing 84, so as to cover the adhesion member 86. Further, in the same manner as in the example illustrated in FIG. 20, an adhesive strength of the housing 84 (electronic apparatus 10) with respect to a body surface may be increased.

### Fifth Embodiment

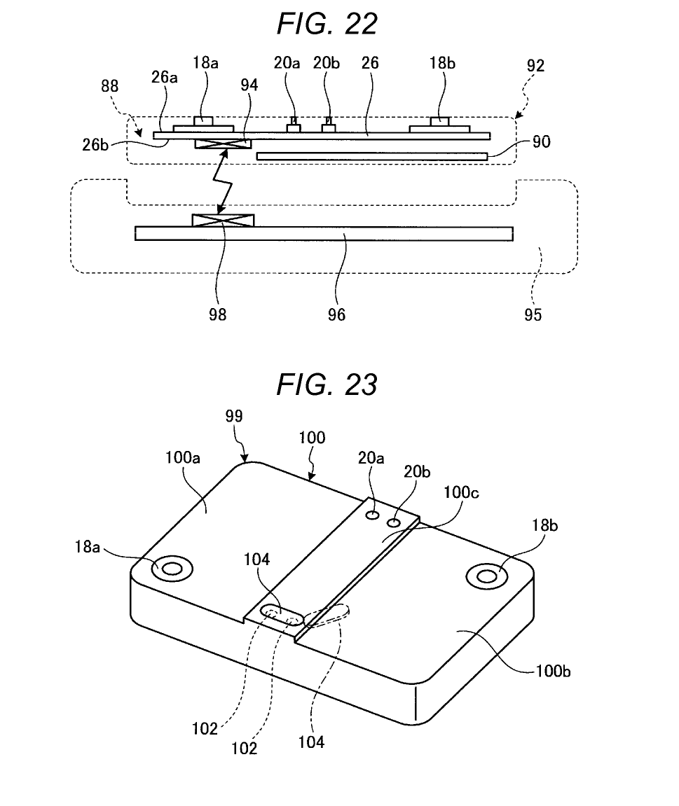

FIG. 22 illustrates an electronic apparatus 92 with a function (wireless charging) of charging a battery 90 that is covered with a housing 88 in a non-contact manner. The electronic apparatus 92 includes a secondary coil 94 mounted on the substrate 26 in order to charge the battery 90 in a non-contact manner, instead of the terminals 22a to 22d for charging that are illustrated in FIG. 3, and the other configurations are the same as those of the electronic apparatus 10 illustrated in FIG. 1 or the like. That is, the electrodes 18a and 18b, and input and output terminals 20a and 20b are mounted on the surface 26a of the substrate 26, and the battery 90, the secondary coil 94, and the plurality of electronic components 30 (not illustrated) are mounted on the back surface 26b. The electronic apparatus 92 configured in this way is placed in a predetermined position of the cradle 95 with a non-contact charging function, and thus a primary coil 98 mounted on the substrate 96 that is buried in the cradle 95 faces the secondary coil 94 of the electronic apparatus 92 at a predetermined interval, and the battery 90 is charged by electromagnetic induction.

In this way, by enabling the electronic apparatus 92 to be charged by a non-contact charging method, a terminal for charging is not required to be exposed in the housing 88, sealing performance of the housing 88 is improved, and thus it is possible to improve waterproof property or dustproof property.

### Sixth Embodiment

An electronic apparatus 99 illustrated in FIG. 23 is a modified example of the electronic apparatus 10 illustrated in FIG. 20. A basic configuration of the electronic apparatus 99 is the same as the electronic apparatus 10 illustrated in FIG. 20. However, the electronic apparatus 99 includes terminals 102 for charging on a surface side on which the electrodes 18a and 18b and the input and output terminals 20a and 20b are exposed. In FIG. 23, the housing 100 includes a first support area portion 100a on which the electrode 18a is exposed and which supports the first adhesion member 80a (refer to FIG. 20), a second support area portion 100b on which the electrode 18b is exposed and which supports the second adhesion member 80b (refer to FIG. 20), and a protrusion portion 100c on which the input and output terminals 20a and 20b are exposed and which includes the terminals for charging 102. Then, the housing 100 includes a cover 104 that covers the terminals 102 and may be opened and closed. That is, only when charging of the electronic apparatus 99 is performed, the cover 104 is opened, the terminals 102 are exposed from the housing 100, whereby charging operation may be performed. Except for this case, the terminals 102 are covered by the cover 104, thereby not being exposed. By configuring in this way, it is possible to gather the electrodes 18a and 18b, the input and output terminals 20a and 20b, the terminals 102, and the like on one surface side of the housing 100. As a result, the rear surface side of the electronic apparatus 99 may be in a sealed state without exposed components, and thus it is possible to improve waterproof property and dustproof property. In addition, since the terminals for charging 102 are sealed by the cover 104, the terminals 102 are not in contact with a body surface and it is possible to improve safety, when a biological signal is detected by the electronic apparatus 99 or the like. In addition, when a biological signal is detected, or the like, a current does not flow through the input and output terminals 20a and 20b, and thus the input and output terminals 20a and 20b may be maintained in a state of being exposed, but in order to protect a terminal surface, another cover that is the same as the cover 104 may be provided.

### Seventh Embodiment

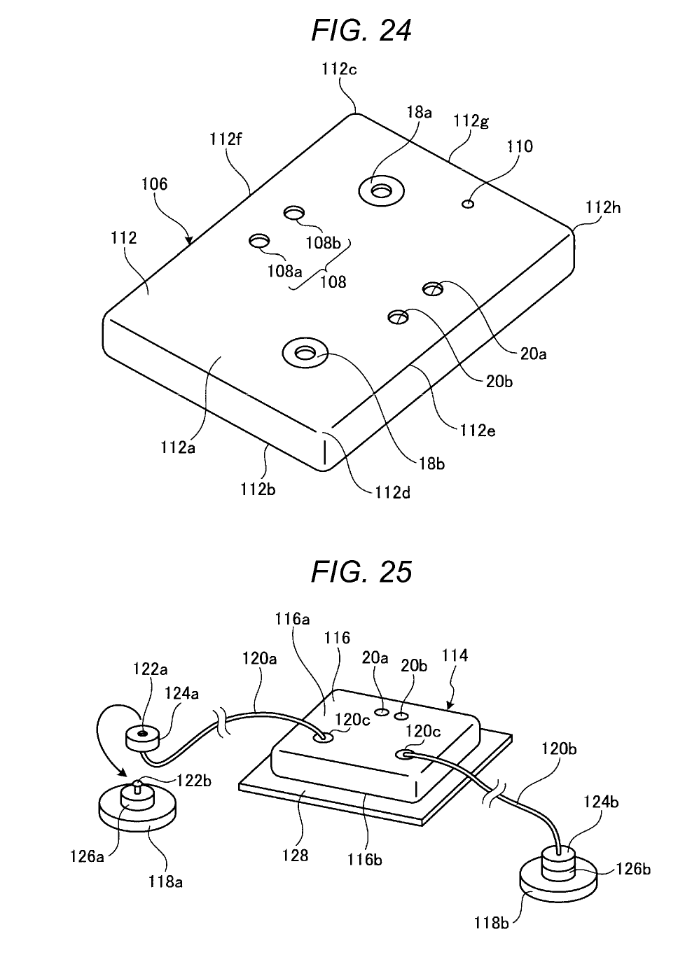

The electronic apparatuses 10, 92, and 99 according to each embodiment described above illustrate apparatuses that detect a biological signal for generating an electrocardiogram as an example. An electronic apparatus 106 illustrated in FIG. 24 includes a pulse wave sensor 108 for measuring a pulse rate of a subject, and a temperature sensor 110 for measuring temperature (body temperature) of the body surface of the subject, in addition to a biological signal for creating an electrocardiogram.

In the same manner as the electronic apparatus 10 illustrated in FIG. 1, a housing 112 that covers the electronic apparatus 106 includes a surface 112a (sensor surface, top surface, first surface, first wall), and a rear surface 112b (charging surface, bottom surface, second surface, second wall). The electrodes 18a and 18b, the input and output terminals 20a and 20b, a pulse wave sensor 108, and temperature sensor 110 are respectively exposed on the surface 112a. The electrode 18a is exposed in the vicinity of a corner 112c of the surface 112a of the housing 112, and the electrode 18b is exposed in the vicinity of a corner 112d of a diagonal position of the corner 112c in the surface 112a. That is, the electrode 18a and the electrode 18b are disposed in a diagonal position such that a distance is largely secured on the housing 112. In addition, the input and output terminals 20a and 20b are disposed substantially in parallel in a position close to a side 112e in a longitudinal direction of the housing 112 on a side close to the electrode 18b.

Meanwhile, in the pulse wave sensor 108, for example, an LED 108a that emits a green light, and a light receiving unit 108b that receives a green light reflected by a body surface of a subject are exposed substantially in parallel with a side 112f, in the vicinity of the side 112f opposing a side 112e. In FIG. 24, the LED 108a and the light receiving unit 108b are exposed in a position opposing the input and output terminals 20a and 20b.

In addition, the temperature sensor 110 is exposed in the vicinity of the other corner 112h of a side 112g, in the vicinity of the side 112g that forms a corner 112c. In addition, the pulse wave sensor 108 and the temperature sensor 110 may be placed in any position in which the sensors may come into contact with a body surface, when the electronic apparatus 106 is mounted on the body surface of a subject. In this way, the electronic apparatus 106 may detect various biological signals at the same time, and may perform integrally a state management or a health management of a subject.

### Eighth Embodiment

An electronic apparatus 114 illustrated in FIG. 25 has a structure in which miniaturization of a housing 116 may be performed more greatly than that of each embodiment described above. Specifically, electrodes 118a and 118b for detecting a biological signal from the electronic apparatus 114 are separated, and are connected to the electronic apparatus 114 by a sensor cable (connection cord) 120a and a sensor cable 120b. One ends of the sensor cable 120a and the sensor cable 120b are introduced from a sealed portion 120c formed on a surface 116a (top surface, first surface, first wall) of the housing 116 to an inside of the housing 116. The sealed portion 120c is a portion of the housing 116, is firmly sealed with a synthetic resin material during insert molding. As a result, waterproof property or dustproof property can be improved, and the sensor cables 120a and 120b can be firmly fixed. On the other end side of the sensor cable 120a, a socket 124a having, for example, a female-type snap electrode 122a that enables attachment and detachment of the electrode 118a and the sensor cable 120a is provided.

In addition, in the electrode 118a, a socket 126a having a male-type snap electrode 122b is provided. In the same manner, on the other end side of the sensor cable 120b, a socket 124b having, for example, a female-type snap electrode 122a (not illustrated) that enables attachment and detachment of the electrode 118b and the sensor cable 120b is provided. In the electrode 118b, a socket 126b having a male-type snap electrode 122b (not illustrated) is provided. In addition, on a detection surface of a biological signal of the electrodes 118a and 118b, a conductive adhesion member (not illustrated) is included in the same manner as in other embodiments, and is configured so as to perform sticking and fixing of the electrodes 118a and 118b to the body surface of a subject. In addition, in the electronic apparatus 114, the electrode 118a and the electrode 118b may be separated from each other by the sensor cable 120a and the sensor cable 120b, and thus it is not required to consider conduction (deformation of viscous member or conduction caused by sweat) of the electrode 118a and the electrode 118b, in the same manner as in the other embodiments.

On the surface 116a of the housing 116, the input and output terminals 20a and 20b are exposed, which are used during transfer of the detected biological signal or update of software, or the like. In addition, on the rear surface 116b of the housing 116, an adhesion member (adhesion sheet) 128 for fixing the housing 116 to a body surface of a subject is pasted. When the pulse wave sensor 108 or the temperature sensor 110 is mounted on the electronic apparatus 114 illustrated in FIG. 24, a detection unit may be separated from the housing 116 using a sensor cable, in the same manner as the electrode 118a (118b). The pulse wave sensor 108 or the temperature sensor 110 may be exposed on the rear surface 116b of the housing 116.

In this way, by separating the electrode 118a and the electrode 118b that are disposed so as to be separated by a distance equal to or longer than a predetermined distance during detection of a biological signal, from the housing 116, a separation distance is not required to be provided on the housing 116 (substrate). As a result, a degree of freedom of layout of a plurality of electronic components is improved, and thus it is possible to further miniaturize the housing 116. In addition, as illustrated in FIG. 25, when detection of a biological signal is performed using the sensor cable 120a or the like, the housing 116 is not required to directly come into contact with a body surface of a subject, and thus a hook for hooking the housing 116 to clothes or the like may be provided in a portion of the housing 116.

### Ninth Embodiment

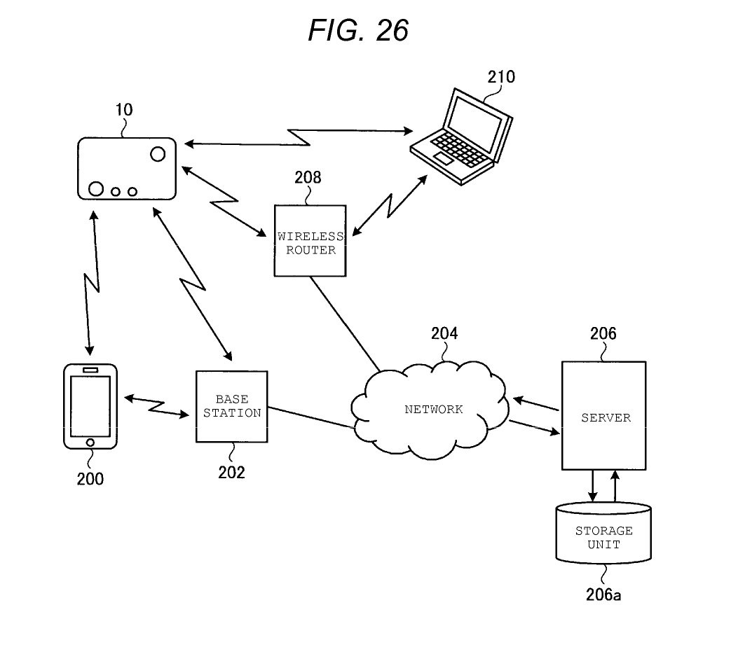

A usage example of the electronic apparatus 10 or the electronic apparatuses 92, 99, 106, and 114 according to each embodiment described above will be described with reference to FIG. 26. For example, when a biological signal (potential, mind potential, detected value) for an electrocardiogram is detected, the electronic apparatus 10 transmits biological information (information, transmission information) obtained based on the detected biological signal toward an external apparatus. The electronic apparatus 10 transfers the biological information (information, transmission information) to a communication terminal 200 (mobile phone, smart phone) carried by a user using a buried communication function, for example, Bluetooth. The communication terminal 200 transmits biological information acquired in a server 206, which is an external apparatus, through a base station 202 and a network 204. In addition, the electronic apparatus 10 may be configured so as to transmit the detected biological signal to the server 206 as it is. In addition, when the electronic apparatus 10 includes a connection function to the network 204, for example, a Wi-Fi communication function, the biological information (biological signal) may be transmitted to the server 206 through the base station 202 and the network 204. In addition, when being able to be connected to a wireless LAN, the electronic apparatus 10 transmits the biological information to the server 206 through a wireless router 208 and the network 204. In addition, the biological information may be transmitted through the wireless router 208 by a personal computer 210 once. In addition, in the example described above, a communication network (electrical communication line) using wireless is described, but a communication network using a wire may be used. In addition, the communication network includes, for example, a router, a modem, an access point, a cable, or the like. In addition, each apparatus may perform data transfer according to a predetermined communication protocol.

The electronic apparatus 10 may transmit the biological information to the server 206 each time being acquired, and may transmit when accumulation of a predetermined amount of signals is completed. In addition, the biological information may be transmitted for each predetermined period, and may be transmitted at timing when a user desires by an operation of the electronic apparatus 10.

When transmitting the biological information to the server 206, the electronic apparatus 10 transmits the biological information together with, for example, a personal ID and a password that are given to each user, in such a manner that an individual may be identified on the server 206 side. In addition, it is also possible to transmit the biological information in a manner that an individual is not specified, using a guest ID.

When acquiring biological information, the server 206 accumulates the biological information in a storage unit 206a, and performs processing according to the biological information. For example, when the biological information indicates an electrocardiographic potential, the server creates an electrocardiogram. Furthermore, the server performs analysis based on the electrocardiogram and generates health condition information. In addition, when the biological information indicates a pulse wave signal or a temperature signal, the server converts the biological information into a pulse wave or temperature, and creates health condition information based on the pulse wave or the temperature. When generating health condition information, the server 206 creates an electrocardiogram based on changes in the biological information of a predetermined period, for example, and creates a change graph of a pulse wave or temperature. In addition, diagnostic information may be created based on the changes. In addition, when a user continually transmits biological information to the server 206 using a personal ID, the server 206 performs changes in a long-term health state or diagnosis, based on a result obtained by comparing an analysis result or diagnostic information of the past with latest analysis result or diagnostic information, and for example, future advice or the like may be created as health state information.

The server 206 accumulates the created health diagnostic information in the storage unit 206a, and retransmits the health diagnostic information to a user that transmits the biological information, through the network 204. For example, when a user transmits the biological information through the communication terminal 200, the health diagnostic information is displayed on a display screen of the communication terminal 200. When a user directly transmits the biological information to the server 206 using a communication function of the electronic apparatus 10, the server 206 transmits the health diagnostic information to the electronic apparatus 10. When receiving the health diagnostic information, the electronic apparatus 10 transfers the health diagnostic information that is received in the communication terminal 200 or the personal computer 210 which is owned by the electronic apparatus, and the health diagnostic information is displayed on the display screen of the communication terminal 200 or the personal computer 210. In the same manner, when the electronic apparatus 10 transmits the biological information to the server 206 through the wireless router 208, the electronic apparatus transmits the health diagnostic information to the personal computer 210 of the user, and may make the health diagnostic information to be displayed on the display screen of the personal computer 210. The health diagnostic information that is transmitted from the server 206 may be retained in the communication terminal 200 or the personal computer 210. In addition, the biological signal that is detected by the electronic apparatus 10 is original data, and may be retained in the communication terminal 200 or the personal computer 210.

In the present embodiment, based on the biological signal detected by the electronic apparatus 10, the biological information is transmitted to the server 206, and thus analyzed. Alternatively, as another embodiment, a dedicated program may be installed in the communication terminal 200 or the personal computer 210, creation of an electrocardiogram or the like, or creation of health diagnostic information may be performed in the communication terminal 200 or the personal computer 210, and the created data may be provided to a user. In addition, the communication terminal 200 or the personal computer 210 may perform a brief analysis or creation of brief health diagnostic information, the server 206 may perform a more detailed analysis or creation of health diagnostic information, according to the needs of a user, and the data may be provided to the user.

As described above, the electronic apparatus according to an embodiment may include the plurality of electrodes that is placed so as to be separated from each other, the electronic component that is electrically connected to the plurality of electrodes, the substrate that supports the plurality of electrodes and the electronic component, and the housing that includes the synthetic resin material which covers the plurality of electrodes, the electronic component, and the substrate, in a state where a portion of the plurality of electrodes is exposed, and the plurality of electrodes, the electronic component, and the substrate are buried. According to this configuration, for example, since the plurality of electrodes, the electronic component, and the substrate that are covered by the housing are respectively surrounded by the synthetic resin material, it is possible to easily obtain waterproof property or dustproof property. In addition, by surrounding the plurality of electrodes, the electronic component, and the substrate with the synthetic resin material, it is possible to mitigate transfer of an impact from outside.

In addition, the housing of the electronic apparatus according to an embodiment may have flexibility. According to this configuration, for example, when the electronic apparatus is mounted on the body surface of a subject, the electronic apparatus may be deformed depending on a shape of the body surface, and it is possible to decrease uncomfortable feeling during mounting. In addition, it is possible to stick the electronic apparatus to the body surface of the subject, and to satisfactorily perform the detection of the biological signal. In addition, hand feeling of the electronic apparatus is improved, and handling is easy.

In addition, on a surface, which is in contact with the housing, of the plurality of electrodes, for example, at least one of the concave portion and the convex portion is provided. According to this configuration, for example, by forming a rough surface caused by the concave portion and the convex portion on the electrodes, when being in contact with the synthetic resin material, the contacted area is increased and thus an anchor effect is exerted. As a result, it is possible to firmly perform bonding between the electrodes and the synthetic resin material.

In addition, on a surface, which is in contact with the housing, of the substrate of the electronic apparatus according to an embodiment, for example, at least one of the concave portion and the convex portion is provided. According to this configuration, for example, by forming a rough surface caused by the concave portion and the convex portion on a surface of the substrate, when being in contact with the synthetic resin material, the contacted area is increased and thus an anchor effect is exerted. As a result, it is possible to firmly perform bonding between the substrate and the synthetic resin material.

In addition, the housing of the electronic apparatus according to an embodiment may include the insert molding, the plurality of electrodes is in contact with a mold during the insert molding, and may support the substrate. According to this configuration, it is possible to correctly and easily position the mold and the substrate during the insert molding using the configuration components of the electronic apparatus. As a result, it is possible to easily stabilize the quality of the electronic apparatus.

In addition, the plurality of electrodes of the electronic apparatus according to an embodiment may include a positioning portion that performs positioning of the substrate with respect to the mold when filling the synthetic resin material in the mold. According to this configuration, it is possible to easily expose the electrode on the surface of the housing that is formed of the synthetic resin material, and to perform correctly and easily positioning of the mold and the electrodes during the insert molding using the configuration components of the electronic apparatus. As a result, it is possible to easily stabilize the quality of the electronic apparatus.

In addition, the electronic apparatus according to an embodiment may have at least a portion that is bendable. According to this configuration, the substrate may be bent depending on the deformation of the housing of the electronic apparatus that is molded by the synthetic resin material. As a result, it is possible to suppress peeling of the electronic components on the substrate, and to easily deform the shape of the electronic apparatus depending on the body surface of the subject.

In addition, the substrate of the electronic apparatus according to an embodiment may include, for example, a first area portion that is bendable, and a second area portion that is more rigid than the first area portion. According to this configuration, it is possible to suppress that the electronic apparatus is unlimitedly deformed, and thus it is possible to suppress peeling of the electronic components on the substrate, and to make the shape of the electronic apparatus correspond to even deformation depending on the body surface of the subject.

In addition, the substrate of the electronic apparatus according to an embodiment may include a first area portion that is configured with flexible printed substrate and is bendable, and a second area portion that is configured with a printed substrate with stronger rigidity than the first area portion which is connected to an end portion of the first area portion. According to this configuration, it is possible to easily form a bendable area and an area in which bending is suppressed. As a result, it is possible to suppress that the electronic apparatus is unlimitedly deformed. In addition, it is possible to suppress peeling of the electronic components on the substrate, and to make the shape of the electronic apparatus correspond to even deformation depending on the body surface of the subject.

In addition, the substrate of the electronic apparatus according to an embodiment is, for example, a flexible printed substrate, and the housing may include a first housing area portion that is bendable, and a second housing area portion with stronger rigidity than the first housing area portion. According to this configuration, it is possible to easily form a bendable area and an area in which bending is suppressed. As a result, it is possible to suppress that the electronic apparatus is unlimitedly deformed. In addition, it is possible to suppress peeling of the electronic components on the substrate, and to make the shape of the electronic apparatus correspond to even deformation depending on the body surface of the subject.

In addition, the substrate of the electronic apparatus according to an embodiment may include the first substrate and the second substrate surface opposite to the first substrate surface, the first substrate surface may support the plurality of electrodes, and the second substrate surface may support the power supply terminal for supplying power to the electronic components. According to this configuration, the components that may be in contact with the body surface of the subject, and the components that are desired not to be in contact with the body surface of the subject are separated from each other and are mounted on the substrate, and thus it is possible to easily achieve safety measure of the electronic apparatus.

In addition, the electronic apparatus according to the present embodiment includes, a plurality of electrodes, an electronic component that is electrically connected to the plurality of electrodes, a substrate that supports the electronic component, and a housing that includes a synthetic resin material which covers the electronic component and the substrate, in a state where the electronic component is in contact with the substrate. The substrate may be connected to each of the plurality of electrodes by the connection code. According to this configuration, since the electronic component and the substrate that are covered by the housing are respectively surrounded by the synthetic resin material, it is possible to easily obtain waterproof property or dustproof property. In addition, by surrounding the electronic component and the substrate using the synthetic resin material, it is possible to mitigate transfer of an impact from outside. At this time, since the electrodes that are required to be disposed with a predetermined interval exist outside the housing, a space necessary for a disposal space or separation distance of the plurality of electrodes in the housing may be reduced, and it is possible to miniaturize the housing, that is, the electronic apparatus.

In addition, the substrate of the electronic apparatus according to the present embodiment may include the coil for charging that charges the battery that supplies power to the electronic apparatus, in a non-contact manner, for example. According to this configuration, it is not necessary to expose the terminal for charging on the housing, and to improve waterproof property or dustproof property of the housing.

In addition, the terminal for charging that charges the battery of the electronic apparatus according to the present embodiment may include a cover that covers the terminal for charging and enables attachment and detachment. According to this configuration, since the terminal for charging may not be exposed on the surface of the housing except when charging, it is possible to contribute to improving safety, and to easily protect the terminal for charging. In addition, it is possible to improve a degree of freedom in disposing the terminal for charging, and to contribute to improving a degree of freedom in layout of the electronic apparatus.

In addition, the plurality of electrodes of the electronic apparatus according to an embodiment may be electrodes that detect the biological signal for an electrocardiogram of a subject, and for example, the substrate may further include a first detector that measures a pulse rate of a subject, and a second detector that measures temperature of the body surface of a subject. According to this configuration, it is possible to simultaneously measure the electrocardiogram, the pulse rate, and body temperature, and to easily perform the integral state management or health management of a subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A measurement device comprising:
a housing;
an electronic unit included in the housing and configured to measure a biological state of a subject; and
an electrode to be placed on the subject for measuring the biological state, wherein
the electrode is embedded in the housing and has a surface that is exposed through an opening in the housing.

2. The measurement device according to claim 1, wherein
the electrode has a first portion and a second portion that has a smaller outer perimeter than the first portion, and
the exposed surface is a surface of the second portion.

3. The measurement device according to claim 1, wherein
the exposed surface is smoother than the other surfaces of the electrode.

4. The measurement device according to claim 1, wherein
the electrode has a concave portion that recedes from the exposed surface.

5. The measurement device according to claim 1, wherein
the exposed surface is flush with the surface of the housing.

6. The measurement device according to claim 1, further comprising:
a second electrode to be placed on the subject for measuring the biological state, wherein the second electrode is electrically connected to the electronic unit and embedded in the housing and has a surface that is exposed through another opening in the housing.

7. The measurement device according to claim 6, wherein
the surface of the housing has a first corner region, a second corner region, a third corner region, and a fourth corner region that is diagonal to the first corner region, and
the first electrode is located closest to the first corner region and the second electrode is located closest to the fourth corner region.

8. The measurement device according to claim 1, further comprising:
a substrate having a first surface on which the electronic unit is disposed and a second surface that is opposite to the first surface and with which the first and second electrodes are in electrical contact.

9. The measurement device according to claim 1, wherein
the electronic unit and the substrate are enclosed entirely within the housing.

10. The measurement device according to claim 1, wherein
the housing is formed of a flexible material.

11. A measurement device comprising:
a housing having an opening;
an electronic unit included in the housing and configured to measure a biological state of a subject; and
an electrode to be placed on the subject for measuring the biological state, wherein the electrode is disposed in the opening of the housing.

12. The measurement device according to claim 11, wherein
a surface of the electrode is exposed to an outside of the measurement device on a surface of the housing.

13. The measurement device according to claim 12, wherein
the electrode has a first portion and a second portion that has a smaller outer perimeter than the first portion, and
the exposed surface is a surface of the second portion.

14. The measurement device according to claim 12, wherein
the exposed surface is smoother than the other surfaces of the electrode.

15. The measurement device according to claim 12, wherein
the electrode has a concave portion that recedes from the exposed surface.

16. The measurement device according to claim 11, wherein
the housing is formed of a flexible material.

17. A method for manufacturing a measurement device for measuring a biological state of a subject, the method comprising:
attaching, on a first surface of a substrate, an electronic unit configured to measure the biological state of the subject;
attaching, on a second surface of a substrate that is opposite to the first surface, an electrode to be placed on the subject for measuring the biological state; and
forming a housing around the substrate, the electronic unit, and the electrode, such that a surface of the electrode is exposed through an opening in the housing.

18. The method according to claim 17, wherein the forming of the housing includes:
placing the substrate to which the electronic unit and the electrode are attached in a mold,
filling the mold with a curable material, and
curing the curable material.

19. The method according to claim 18, wherein
the substrate is placed such that the surface of the electrode contacts a wall of the mold.

20. The method according to claim 19, wherein
the substrate includes a protrusion that contacts a wall of the mold that is opposite to the wall that contacts the electrode when the substrate is placed in the mold.
